(19) United States
(12) Patent Application Publication
(10) Pub. No.: US 2023/0323435 A1
(43) Pub. Date: Oct. 12, 2023

(54) METHODS AND COMPOSITIONS FOR MAXIMUM RELEASE OF OLIGONUCLEOTIDES

(71) Applicant: Bio-Rad Laboratories, Inc., Hercules, CA (US)

(72) Inventors: Vishal SHARMA, Irvine, CA (US); Ronald LEBOFSKY, Berkeley, CA (US)

(21) Appl. No.: 18/185,220

(22) Filed: Mar. 16, 2023

Related U.S. Application Data
(60) Provisional application No. 63/321,266, filed on Mar. 18, 2022.

(51) Int. Cl. C12Q 1/6837; C12N 15/10

(52) U.S. Cl. CPC C12Q 1/6837; C12N 15/1065

(57) ABSTRACT

The methods allow for provision of a mixture of a plurality of beads, each bead linked to oligonucleotides, wherein the mixture can be treated as a bulk solution (prior to partitioning) to cleave a covalent bond linking the oligonucleotides to the beads while retaining a non-covalent linkage (via hybridization) between the beads and the oligonucleotides, allowing for distribution of the oligonucleotides and beads to partitions or 2D arrays prior to separation of the oligonucleotides from the beads, which occurs for example in the partitions.

### CROSS-REFERENCES TO RELATED APPLICATIONS

This application claims the benefit of and priority to U.S. Provisional Patent Application 63/321,266, filed on Mar. 18, 2022, which is hereby incorporated by reference in its entirety for all purposes.

### SEQUENCE LISTING

A Sequence Listing conforming to the rules of WIPO Standard ST.26 is hereby incorporated by reference. The Sequence Listing has been filed as an electronic document via PatentCenter in ASCII format encoded as XML. The electronic document, created on Mar. 15, 2023, is entitled “094868-1373690-119610US-ST26.xml”, and is 30,910 bytes in size.

### BACKGROUND OF THE INVENTION

Tagging biological substrates with molecular barcodes in partitions can provide novel biological insight of the substrates that co-localize to discrete partitions, through the sequencing of the molecular barcodes and analysis, thereof. Increasing the number of barcoding competent partitions, such as droplets, increases the number of sequencing based data points and converts a greater fraction of input substrates into data. Barcodes can be delivered to partitions, such as droplets, using beads as the delivery vehicle. In order to uniquely identify each partition, the beads can be labeled with clonal copies of unique barcode sequences, which can be released into the partition to tag molecules in the partition in a partition-specific manner.

### BRIEF SUMMARY OF THE INVENTION

In some embodiments, a method of releasing an oligonucleotide from a bead is provided. In some embodiments, the method comprises,
(i) providing a reaction mixture comprising:
a plurality of beads, each bead covalently linked to a first oligonucleotide comprising a first end sequence,
a second oligonucleotide comprising a second end sequence; and
a linking oligonucleotide comprising (i) a first terminal sequence that is reverse complementary to the first end sequence and (ii) a second terminal sequence that is reverse complementary to the second end sequence,
wherein the first terminal sequence and first end sequence are hybridized and have a first melting temperature (Tm) and (ii) the second terminal sequence and second end sequence are hybridized and have a second Tm such that the linking oligonucleotide links the first oligonucleotide to the second oligonucleotide;
(ii) raising the temperature of the reaction mixture higher than at least one of the first and second Tm such that the first oligonucleotide and the second oligonucleotide are disassociated from at least one end of the linking oligonucleotide;
(iii) lowering the temperature of the reaction mixture below the first and second Tm,
wherein after the raising and before the lowering the reaction mixture further comprises a blocking oligonucleotide comprising either:
(a) a sequence that is reverse complementary to the first end sequence but does not comprise a sequence of more than 3 contiguous nucleotides reverse complementary to the second end sequence, such that the blocking oligonucleotide competes with the linking oligonucleotide for hybridization to the first oligonucleotide, allowing the second oligonucleotide to remain released from the bead, or
(b) a sequence that is reverse complementary to the second end sequence but does not comprise a sequence of more than 3 contiguous nucleotides reverse complementary to the first end sequence, such that the blocking oligonucleotide competes with the linking oligonucleotide for hybridization to the second oligonucleotide, allowing the second oligonucleotide to remain released from the bead or
(c) a sequence that is the first end sequence but does not comprise a sequence of more than 3 contiguous nucleotides in the second end sequence, such that the blocking oligonucleotide competes with the linking oligonucleotide for hybridization to the first oligonucleotide, allowing the second oligonucleotide to remain released from the bead, or
(d) a sequence that is the second end sequence but does not comprise a sequence of more than 3 contiguous nucleotides that is the first end sequence, such that the blocking oligonucleotide competes with the linking oligonucleotide for hybridization to the second oligonucleotide, allowing the second oligonucleotide to remain released from the bead.

In some embodiments, the first end sequence is a 3′ end sequence. In some embodiments, the first end sequence is a 5′ end sequence.

In some embodiments, the second oligonucleotide has a barcode sequence, wherein individual beads comprise clonal copies of the second oligonucleotide and wherein the barcode sequence for individual beads are unique such that the barcode distinguishes the bead from other beads in the plurality. In some embodiments, the 3′ end of the second oligonucleotide comprises a target-specific sequence. In some embodiments, the 3′ end of the second oligonucleotide comprises a universal tag sequence. In some embodiments, the 3′ end of the second oligonucleotide comprises at least 4 contiguous thymines.

In some embodiments, the providing (i) comprises forming a mixture of beads, wherein each bead is covalently linked to a long oligonucleotide comprising the first oligonucleotide and the second oligonucleotide, wherein the first end sequence of the first oligonucleotide is linked directly, or indirectly via a linker sequence, to the second end sequence of the second oligonucleotide, and wherein long oligonucleotides on different beads are distinguishable by a different barcode sequence in the long oligonucleotide; and hybridizing the linking oligonucleotide to the long oligonucleotide such that the first terminal sequence is hybridized to the first end sequence and the second terminal sequence is hybridized to the second end sequence; and cleaving the long oligonucleotide between the first end sequence and the second end sequence while the linking oligonucleotide remains intact and links the first oligonucleotide to the second oligonucleotide. In some embodiments, the linker sequence comprises one or more uracil nucleotide and the cleaving comprises contacting the long oligonucleotide with uracil DNA glycosylase and endonuclease VIII, thereby excising the one or more uracil. In some embodiments, the linker sequence comprises one or more ribonucleotide and the cleaving comprises cleaving the linker sequence in a ribonucleotide-specific manner using RNAseH. In some embodiments, a restriction site is located between the first oligonucleotide and the second oligonucleotide and the cleaving comprises contacting the long oligonucleotide with a restriction enzyme that cleaves the restriction site on the long oligonucleotide without cleaving the linking oligonucleotide using a nicking endonuclease.

In some embodiments, the blocking oligonucleotide is added to the reaction mixture following the cleaving of the long oligonucleotide between the first end sequence and the second end sequence.

In some embodiments, the concentration of the blocking oligonucleotide in the reaction mixture is higher than the concentration of the linker oligonucleotide in the reaction mixture. In some embodiments, the affinity (Kd) of the blocking oligonucleotide for the first sequence is lower than the affinity of the linker oligonucleotide for the first sequence.

In some embodiments, the method further comprises distributing the reaction mixture into a plurality of partitions after the providing (i) and before the raising (ii), wherein different beads of the plurality are delivered into different partitions. In some embodiments, the partitions are microwells, nanowells or droplets.

In some embodiments, the method further comprises distributing the reaction mixture onto a 2D array after the providing (i) and before the raising (ii), wherein different beads of the plurality are delivered onto different locations on the 2D array.

In some embodiments, a method of forming a cleaved oligonucleotide linked to a bead is provided. In some embodiments, the method comprises: forming a mixture of beads, wherein each bead is covalently linked to a long oligonucleotide comprising a first oligonucleotide and a second oligonucleotide, wherein a first end sequence of the first oligonucleotide is linked directly, or indirectly via a linker sequence, to a second end sequence of the second oligonucleotide, and wherein long oligonucleotides on different beads are distinguishable by a different barcode sequence in the long oligonucleotide; and hybridizing a linking oligonucleotide to the long oligonucleotide, wherein the linking oligonucleotide comprises (i) a first terminal sequence that is reverse complementary to the first end sequence and (ii) a second terminal sequence that is reverse complementary to the second end sequence, wherein the hybridizing results in the first terminal sequence hybridized to the first end sequence and the second terminal sequence hybridized to the second end sequence; and cleaving the long oligonucleotide between the first end sequence and the second end sequence while the linking oligonucleotide remains intact and links the first oligonucleotide to the second oligonucleotide.

In some embodiments, linker sequence comprises one or more uracil nucleotide and the cleaving comprises contacting the long oligonucleotide with uracil DNA glycosylase and endonuclease VIII, thereby excising the one or more uracil. In some embodiments, the linker sequence comprises one or more ribonucleotide and the cleaving comprises cleaving the linker sequence in a ribonucleotide-specific manner using RNAseH. In some embodiments, a restriction site is located between the first oligonucleotide and the second oligonucleotide and the cleaving comprises contacting the long oligonucleotide with a restriction enzyme that cleaves the restriction site on the long oligonucleotide without cleaving the linking oligonucleotide using a nicking endonuclease.

In some embodiments, a blocking oligonucleotide is added to the reaction mixture following the cleaving of the long oligonucleotide between the first end sequence and the second end sequence.

Also provided is a reaction mixture comprising, e.g., a plurality of beads, each bead covalently linked to a first oligonucleotide comprising a first end sequence, a second oligonucleotide comprising a second end sequence; and a linking oligonucleotide comprising (i) a first terminal sequence that is reverse complementary to the first end sequence and (ii) a second terminal sequence that is reverse complementary to the second end sequence, and a blocking oligonucleotide comprising either:
(a) a sequence that is reverse complementary to the first end sequence but does not comprise a sequence of more than 3 contiguous nucleotides reverse complementary to the second end sequence, such that the blocking oligonucleotide competes with the linking oligonucleotide for hybridization to the first oligonucleotide, allowing the second oligonucleotide to remain released from the bead, or
(b) a sequence that is reverse complementary to the second end sequence but does not comprise a sequence of more than 3 contiguous nucleotides reverse complementary to the first end sequence, such that the blocking oligonucleotide competes with the linking oligonucleotide for hybridization to the second oligonucleotide, allowing the second oligonucleotide to remain released from the bead or
(c) a sequence that is the first end sequence but does not comprise a sequence of more than 3 contiguous nucleotides in the second end sequence, such that the blocking oligonucleotide competes with the linking oligonucleotide for hybridization to the first oligonucleotide, allowing the second oligonucleotide to remain released from the bead, or
(d) a sequence that is the second end sequence but does not comprise a sequence of more than 3 contiguous nucleotides that is the first end sequence, such that the blocking oligonucleotide competes with the linking oligonucleotide for hybridization to the second oligonucleotide, allowing the second oligonucleotide to remain released from the bead.

In some embodiments, the first end sequence is a 3′ end sequence. In some embodiments, the first end sequence is a 5′ end sequence.

In some embodiments, the second oligonucleotide has a barcode sequence, wherein individual beads comprise clonal copies of the second oligonucleotide and wherein the barcode sequence for individual beads are unique such that the barcode distinguishes the bead from other beads in the plurality. In some embodiments, the 3′ end of the second oligonucleotide comprises a target-specific sequence. In some embodiments, the 3′ end of the second oligonucleotide comprises a universal tag sequence. In some embodiments, the 3′ end of the second oligonucleotide comprises at least 4 contiguous thymines.

In some embodiments, the linker sequence comprises one or more uracil nucleotide In some embodiments, the linker sequence comprises one or more ribonucleotide. In some embodiments, a restriction site is located between the first oligonucleotide and the second oligonucleotide.

Also provided is a kit (e.g., a container, optionally with instructions) comprising the plurality of beads as described above or elsewhere herein.

Also provided is a reaction mixture comprising: a plurality of beads, each bead covalently linked to a first oligonucleotide comprising a first end sequence, a second oligonucleotide comprising a second end sequence; and a linking oligonucleotide comprising (i) a first terminal sequence that is reverse complementary to the first end sequence and (ii) a second terminal sequence that is reverse complementary to the second end sequence, wherein the first terminal sequence and first end sequence are hybridized and have a first melting temperature (Tm) and (ii) the second terminal sequence and first end sequence are hybridized and have a second Tm such that the linking oligonucleotide links the first oligonucleotide to the second oligonucleotide; wherein the reaction mixture optionally comprises a blocking oligonucleotide comprising either:
(a) a sequence that is reverse complementary to the first end sequence but does not comprise a sequence of more than 3 contiguous nucleotides reverse complementary to the second end sequence, such that the blocking oligonucleotide competes with the linking oligonucleotide for hybridization to the first oligonucleotide, allowing the second oligonucleotide to remain released from the bead, or
(b) a sequence that is reverse complementary to the second end sequence but does not comprise a sequence of more than 3 contiguous nucleotides reverse complementary to the first end sequence, such that the blocking oligonucleotide competes with the linking oligonucleotide for hybridization to the second oligonucleotide, allowing the second oligonucleotide to remain released from the bead or
(c) a sequence that is the first end sequence but does not comprise a sequence of more than 3 contiguous nucleotides in the second end sequence, such that the blocking oligonucleotide competes with the linking oligonucleotide for hybridization to the first oligonucleotide, allowing the second oligonucleotide to remain released from the bead, or
(d) a sequence that is the second end sequence but does not comprise a sequence of more than 3 contiguous nucleotides that is the first end sequence, such that the blocking oligonucleotide competes with the linking oligonucleotide for hybridization to the second oligonucleotide, allowing the second oligonucleotide to remain released from the bead.

In some embodiments, the first end sequence is a 3′ end sequence. In some embodiments, the first end sequence is a 5′ end sequence.

In some embodiments, the second oligonucleotide has a barcode sequence, wherein individual beads comprise clonal copies of the second oligonucleotide and wherein the barcode sequence for individual beads are unique such that the barcode distinguishes the bead from other beads in the plurality In some embodiments, the 3′ end of the second oligonucleotide comprises a target-specific sequence. In some embodiments, the 3′ end of the second oligonucleotide comprises a universal tag sequence. In some embodiments, the 3′ end of the second oligonucleotide comprises at least 4 contiguous thymines.

Also provided is a kit comprising the plurality of beads of as described above.

Also provided is a mixture comprising a plurality of beads, wherein each bead is covalently linked to a hairpin oligonucleotide comprising 5′ to 3′ a reverse complement of a first sequence, a loop sequence, a first copy of the first sequence, and a second sequence, wherein the reverse complement of the first sequence is hybridized to the first copy of the first sequence, and the second sequence is at the 3′ end of the hairpin oligonucleotide, wherein a cleavable sequence is located in the reverse complement of the first sequence, in the loop sequence, or in the first copy of the first sequence, wherein the first sequence has a barcode sequence, wherein individual beads comprise clonal copies of the first sequence and wherein the barcode sequence for individual beads are unique such that the barcode distinguishes the bead from other beads in the plurality.

In some embodiments, the loop sequence further comprises a second copy of the first sequence located 5′ of the cleavable sequence. In some embodiments, the 3′ end of the second oligonucleotide comprises a target-specific sequence. In some embodiments, the 3′ end of the second oligonucleotide comprises a universal tag sequence. In some embodiments, the 3′ end of the second oligonucleotide comprises at least 4 contiguous thymines.

In some embodiments, the cleavable sequence comprises one or more uracil nucleotide In some embodiments, the cleavable sequence comprises one or more ribonucleotide. In some embodiments, the cleavable sequence comprises a restriction site.

Also provided is a kit comprising the plurality of beads as described above.

Also provided is a method of forming a plurality of released oligonucleotides. In some embodiments, the method comprises, providing the mixture as described above or elsewhere herein and cleaving the cleavable sequence 3′ of the second copy of the first sequence, while the first copy of the first sequence and the reverse complement of the first sequence remain hybridized, linking the first copy of the first sequence to the bead via the hybridization.

In some embodiments, the cleavable sequence comprises one or more uracil nucleotide and the cleaving comprises contacting the long oligonucleotide with uracil DNA glycosylase and endonuclease VIII, thereby excising the one or more uracil. In some embodiments, the cleavable sequence comprises one or more ribonucleotide and the cleaving comprises cleaving the linker sequence in a ribonucleotide-specific manner using RNAseH. In some embodiments, the cleavable sequence comprises a restriction site and the cleaving comprises contacting a nicking endonuclease to the cleavable sequence.

In some embodiments, the method further comprises distributing the reaction mixture into a plurality of partitions, wherein different beads of the plurality are delivered into different partitions. In some embodiments, the partitions are microwells, nanowells or droplets. In some embodiments, the method further comprises distributing the reaction mixture onto a 2D array after the providing (i) and before the raising (ii), wherein different beads of the plurality are delivered onto different locations on the 2D array.

In some embodiments, the loop sequence further comprises a second copy of the first sequence located 5′ of the cleavable sequence and wherein the first copy of the first sequence and the reverse complement of the first sequence when hybridized, have a melting temperature (Tm), and the method further comprises after the distributing, (ii) raising the temperature of the reaction mixture higher than the Tm such that the first copy of the first sequence and reverse complement of the first sequence are disassociated; (iii) lowering the temperature of the reaction mixture below the Tm, wherein the second copy of the first sequence competes with the first copy of the first sequence for hybridization to the reverse complement of the first sequence, allowing an oligonucleotide comprising the first copy of the first sequence and the second sequence to remain released from the bead after the lowering.

In some embodiments, the first copy of the first sequence and the reverse complement of the first sequence when hybridized, have a melting temperature (Tm), and the method further comprises after the distributing, (ii) raising the temperature of the reaction mixture higher than the Tm such that the first copy of the first sequence and reverse complement of the first sequence are disassociated; (iii) lowering the temperature of the reaction mixture below the Tm, wherein after the raising and before the lowering the mixture further comprises a blocking oligonucleotide comprising a second copy of the first sequence, such that the blocking oligonucleotide competes with the first copy of the first sequence for hybridization to the first oligonucleotide, allowing an oligonucleotide comprising the first copy of the first sequence and the second sequence to remain released from the bead after the lowering.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “amplification reaction” refers to any in vitro means for multiplying the copies of a target sequence of nucleic acid in a linear or exponential manner. Such methods include but are not limited to two-primer methods such as polymerase chain reaction (PCR); ligase methods such as DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are 0, or fewer than 2 or 3 complementarity mismatches over at least about 12, 14, 16, 18, or 20 contiguous nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, adjacent to a primer hybridization site, or flanked by a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full-length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel or microwell. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil). Exemplary array of wells and well descriptions can be found for example in U.S. Pat. Nos. 9,103,754 and 10,391,493. The array of wells (set of nanowells, microwells, wells) can function to capture the solid supports, optionally in addressable, known locations. As such, the array of wells can be configured to facilitate bead capture in at least one of a single-solid support format or optionally in small groups of solid supports. Exemplary microwell arrays and methods of delivery of beads to the microwells and analysis thereof is described in, e.g., PCT/US2021/034152.

As used herein a “barcode” is a short nucleotide sequence (e.g., at least about 4, 6, 8, 10, 12, 15, 20, 50 or 75 or 100 nucleotides long or more) that identifies a molecule to which it is conjugated or from the partition in which it originated. Barcodes can be used, e.g., to identify molecules originating in a partition, bead, or spot as later sequenced from a bulk reaction. Such a barcode can be unique for that partition, bead or spot as compared to barcodes present in other partitions, bead or spot. For example, partitions containing target RNA from single-cells can be subject to reverse transcription conditions using primers that contain different partition-specific barcode sequence in each partition, thus incorporating a copy of a unique “cellular barcode” (because different cells are in different partitions and each partition has unique partition-specific barcodes) into the reverse transcribed nucleic acids of each partition. Thus, nucleic acid from each cell can be distinguished from nucleic acid of other cells due to the unique “cellular barcode.” In some embodiments described herein, barcodes described herein uniquely identify the molecule to which it is conjugated, i.e., the barcode acts as a unique molecular identifier (UMI). The length of the underlying barcode sequence determines how many unique samples can be differentiated. For example, a 1 nucleotide barcode can differentiate 4, or fewer depending on degeneracy, different partitions; a 4 nucleotide barcode can differentiate 4⁴ or 256 partitions or less; a 6 nucleotide barcode can differentiate 4096 different partitions or less; and an 8 nucleotide barcode can index 65,536 different partitions or less.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

An “oligonucleotide” is a polynucleotide. Generally oligonucleotides will have fewer than 250 nucleotides, in some embodiments, between 4-200, e.g., 10-150 nucleotides.

“Clonal” copies of a polynucleotide means the copies are identical in sequence. In some embodiments, there are at least 100, 1000, 10⁴ or more clonal copies of oligonucleotides in linked to a bead.

An “array” is an ordered plurality of items. The term can refer to an ordered plurality of oligonucleotides, or the ordered array linked to a solid surface that is optionally planar. “ordered” refers to known locations on the array and is not intended to indicate a particular alignment of the items, though in some embodiments the items can be in a grid.

A “3′ capture sequence” on an oligonucleotide refers to the 3′ most portion of an oligonucleotide. The capture sequence can be as few as 1-2 nucleotides in length but is more commonly 6-12 nucleotides in length and in some embodiments is 4-20 or more nucleotides in length. The capture sequence can be completely complementary to a target nucleic acid (e.g., the 3′ end of the target nucleic acid), though as will be appreciated in some embodiments and certain conditions, 1, 2, 3, 4, or more nucleotides may be mismatched while still allowing the 3′ capture sequence of an oligonucleotide anneal to the target nucleic acid. In other embodiments, conditions can be selected such that only completely complementary sequences will anneal. The 3′ capture sequence can be a random sequence, a poly T or poly A sequence, a target-specific sequence, or a universal sequence. For example, in embodiments in which a transposon (e.g., a modified Tn5 such as tagmentase) inserts an end sequence to sample nucleic acids, the capture 3′ end sequence can be complementary to the added end sequence.

“Tagging” a nucleic acid refers to linking the nucleic acid with another tagging polynucleotide, for example a tagging polynucleotide comprising one or more barcode sequences. Tagging can be covalent (e.g., via ligation or by primer extension) or non-covalent (via Watson-Crick base pairing only).

The term “bead” refers to any solid support that can be in a partition, e.g., a small particle or other solid support. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

As used herein, a loop sequence in a hairpin oligonucleotide refers to the portion of the oligonucleotide that is not reverse complementary to other sequences in the oligonucleotide and is between two reverse complementary portions. See, e.g., poly T portion depicted on the right side of FIG. 4A.

### BRIEF DESCRIPTION OF THE DRAWINGS

FIG. 1 depicts an illustration of a “long” oligonucleotide linked to a bead (top portion of figure), wherein the long oligonucleotide comprises the first oligonucleotide and the second oligonucleotide sequence separate by a cleavable sequence, which in the case depicted is a series of uracils that can be cleaved by USER enzymes. The bottom portion of the figure depicts the same long oligonucleotide sequence hybridized to a linking oligonucleotide that hybridizes to portions of the first and second oligonucleotide sequences. As used herein, the term “oligonucleotide” can refer to the sequence of the oligonucleotide as part of a longer nucleic acid.

FIG. 2 depicts the same molecules as shown in FIG. 1, but followed by cleavage with the USER enzymes to result in the first oligonucleotide linked to the bead but cleaved from the second oligonucleotide. However, the linking oligonucleotide remains annealed to both the first and second oligonucleotides preventing the two parts to diffuse away from each other. As depicted in the figure, a blocking oligonucleotide is added to the mixture after cleaving of the long oligonucleotide, though as discussed herein when the blocking oligonucleotide is added can vary. The blocking oligonucleotide can be provided at a higher concentration than the linking oligonucleotide and/or can otherwise be modified to have higher affinity than the linking oligonucleotide such that following raise and lowering of the temperature the blocking oligonucleotide is a better competitor than the linking oligonucleotide for at least one site to which the linking oligonucleotide hybridizes. All of the actions depicted in FIG. 2 can occur in a bulk solution, i.e., prior to moving the beads and oligonucleotides linked thereto, into partitions or 2D arrays.

FIG. 3 proceeds from FIG. 2 and depicts the oligonucleotides noted above following distribution into partitions (e.g., droplets or microwells) or separated onto a 2D array. Because the linking oligonucleotide links the now-cleaved oligonucleotides to the beads, one can deliver beads to the partitions or 2D array, already cleaved but nevertheless non-covalently linked. Once in the partitions, raising and lowering the temperature will release the second oligonucleotide from the first oligonucleotide via disassociation of the linking oligonucleotide from one or more of the first and second oligonucleotide, while the blocking oligonucleotide, through its superior competition, prevents re-annealing of the linking oligonucleotide to at least one of the first and second oligonucleotide, allowing the second oligonucleotide to be free in solution, which allows for improved effect of the second oligonucleotide, e.g., as a primer or probe for target nucleic acids.

FIGS. 4A to 4C depict a hairpin oligonucleotide embodiment in which the first and second oligonucleotide are linked to the beads via a loop sequence and a reverse complement of the first oligonucleotide sequence. FIG. 4A depicts an embodiment in which the cleavable sequence (UUU) is located 5′ of the loop sequence. FIG. 4B depicts an embodiment in which the cleavable sequence (UUU) is in the loop sequence. FIG. 4C depicts the said embodiment in which the cleavable sequence (UUU) is located 3′ of the loop sequence.

FIGS. 5A to 5B depict an alternative hairpin oligonucleotide embodiment in which the first and second oligonucleotide are linked to the bead via a variable length sequence, a loop, a reverse complement of the variable length sequence, and a reverse complement of the first oligonucleotide sequence. FIG. 5A depicts an embodiment in which the cleavable sequence (UUU) is located 3′ of the variable length sequence. FIG. 5B depicts an embodiment in which the cleavable sequence (UUU) is located 5′ of the reverse complement of the variable length sequence

FIGS. 6A to 6C depict an alternative hairpin oligonucleotide embodiment in which the first and second oligonucleotide are linked to the beads via a loop sequence, which contains a second copy of the first oligonucleotide, and a reverse complement of the first oligonucleotide sequence. FIG. 6A depicts an embodiment in which the cleavable sequence (UUU) is located 3′ of the loop sequence. Cleavage of the UUU motif is depicted, followed by intramolecular hybridization between the second copy of the first oligonucleotide sequence and the reverse complement of the first oligonucleotide sequence and release of the first and second oligonucleotide sequences that are 3′ of the cleavable sequence. FIG. 6B depicts an embodiment in which the cleavable sequence (UUU) is located within the loop sequence, but 3′ of the second copy of the first oligonucleotide. FIG. 6C depicts the embodiment in FIG. 6A in which a short sequence is located 5′ of the cleavable sequence (UUU) and a reverse complement of the short sequence is located 5′ of the loop sequence.

FIG. 7 depicts a “long” oligonucleotide linked to a bead (top portion of figure), wherein the long oligonucleotide comprises the first oligonucleotide and the second oligonucleotide sequence separated by a cleavable sequence, which in the case depicted is a series of uracils that can be cleaved by USER enzymes. The stitch bead base oligonucleotide refers to the sequence that is not variable between beads. Blocks 1, 2, and 3 refer to 3 variable sequences that comprise the barcode sequences that are clonal for beads, but different between beads. Bioanalyzer traces are shown from experiments where beads in bulk were subjected to various conditions. After +/−hybridization with the linker sequence, “RC, +/−USER cleavage, washing the beads, and +/−heat, the supernatant was collected and run on a gel. Release of the full length oligonucleotide 2 sequence was dependent on the use of RC, USER, and heat.

FIG. 8 depicts Stitch oligonucleotide release compared to USER reagent. Splint oligos (Tm 62 C) were annealed to stitch beads, followed by USER digest in bulk. Then, the beads were aliquoted and incubated at different temperatures. A sample was taken from the supernatant of each aliquot at different timepoint for ddPCR. As a reference, total number of oligos on these beads that were quantified by ddPCR after 1 hr of USER digest (no splint) was also included in the graph.

FIG. 9 depicts a comparison of stitch beads vs USER digested beads in a simultaneous protocol as described in Example 3.

FIG. 10 depicts a comparison of stitch beads vs USER digested beads as described in Example 4.

### DETAILED DESCRIPTION OF THE INVENTION

### Introduction

The present disclosure provides methods and compositions to efficiently achieve partitioning of bead-linked barcoded oligonucleotides wherein the barcoded oligonucleotides are readily released from bead once in partitions. The methods allow for provision of a mixture of a plurality of beads, each bead linked to oligonucleotides, wherein the mixture can be treated as a bulk solution (prior to partitioning) to cleave a covalent bond linking the oligonucleotides to the beads while retaining a non-covalent linkage (via hybridization) between the beads and the oligonucleotides, allowing for distribution of the oligonucleotides and beads to partitions or 2D arrays prior to separation of the oligonucleotides from the beads, which occurs for example in the partitions.

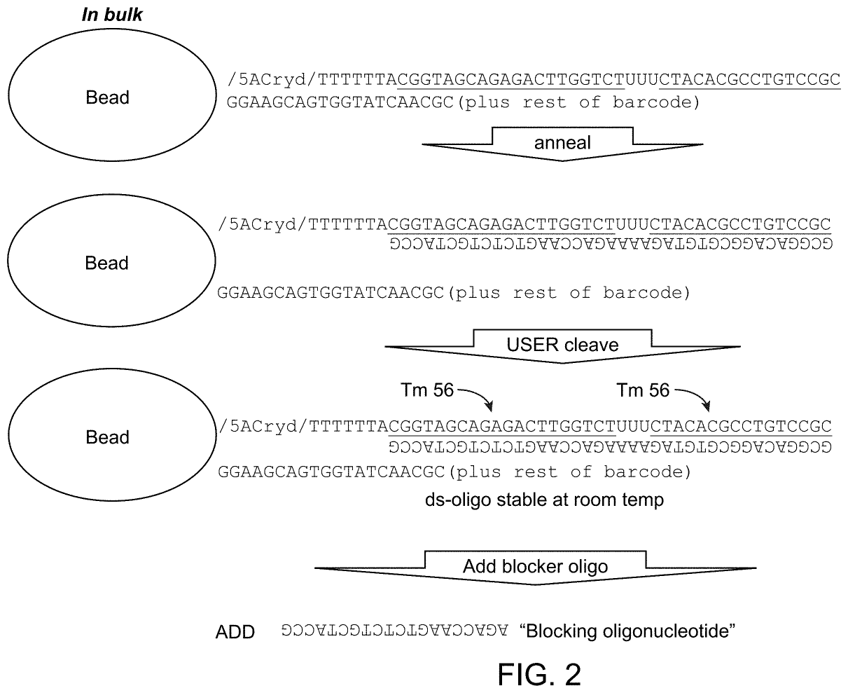

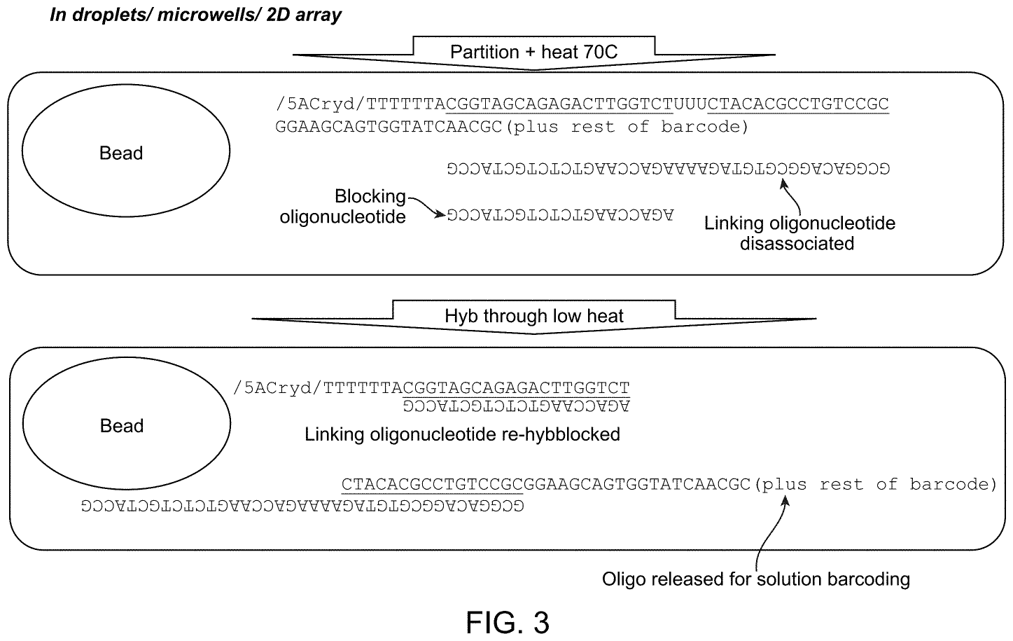

In some embodiments, the methods described herein involve providing an oligonucleotide covalently linked to a bead that includes a cleavable site on a first strand and a second strand (e.g., a linking oligonucleotide) that is complementary to sequences on the first strand that are adjacent on both sides to the cleavable site such that following cleavage (e.g., nicking) of the first strand at the cleavable site the second strand continues to link the oligonucleotide to the bead via hybridization of the second strand to the two segments of the first strand as generated by the cleavage. See, e.g., FIG. 2. In these embodiments, the second strand can be independent (separate from) the first strand, e.g., such that if the first and second strands were disassociated, the first and second strands would no longer be linked in any way. In other embodiments, the methods described herein involve providing a hairpin oligonucleotide covalently linked to a bead that includes a loop sequence that includes a cleavable site wherein a reverse complement of a first sequence, which is linked to the bead, is separated by the loop sequence from the first sequence, and optionally a further second sequence. In these embodiments, following cleavage of the cleavable site, the first (and if included second) sequence remain non-covalently linked to the reverse complement of a first sequence via hybridization until distributed to partitions or a 2D array.

In any of the embodiments described above, once distributed to partitions or a 2D array, the temperature can be raised to disassociate hybridized sequences, releasing the oligonucleotide from the “stub” sequence remaining on the bead. To prevent re-annealing to the stub sequence on the bead once the temperature is dropped, a blocker oligonucleotide sequence is provided in the partition to act as a competitor to prevent re-hybridization by the released oligonucleotide to the stub sequence or alternatively to the linking oligonucleotide. The blocker can be an independent molecule from the oligonucleotide, or in some embodiments of the hairpin constructs, can be included as part of the hairpin sequence itself. Another way to disassociate hybridized sequences is to use basic pH solutions to dissociate the strands, followed by neutralization to allow for hybridization of the blocking oligonucleotide and subsequent use of the released portion of the oligonucleotide to the target nucleic acid. For example, on a 2D array, basic pH denaturation of the second oligonucleotide can be performed followed by addition of pH-neutralizing buffer to allow for hybridization and extension of the release oligonucleotide, for example, to barcode the target nucleic acid. In another example, basic solutions can be delivered to droplets by co-flowing a basic solution during droplet formation, followed by neutralization through the addition, for example by microfluidic methods, of a neutralizing reagent or solution into the droplets. Methods and compositions for delivering reagents to one or more partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

As noted above, an advantage of the embodiments described herein is that oligonucleotides linked to beads can be generated in bulk, treated by the methods described herein in bulk, and then be distributed into partitions or otherwise distributed in a 2D array, at which point the oligonucleotides can be disassociated from the beads. As can be seen, by removing the covalent linkage of the oligonucleotide from the bead (a portion of the oligonucleotide is left on the bead), one can still deliver the oligonucleotides covalently linked to the beads to partitions or a 2D array, without having to cleave a covalent bond within partitions or a 2D array. This is beneficial at least because methods of covalent bond cleavage in partitions or a 2D array can introduce inefficiencies.

The first and second nucleic acid strands can be provided as separate strands or they can be covalently-linked via a hairpin sequence. In some embodiments described herein, the methods involve using the oligonucleotides non-covalently linked (i.e., having been nicked as described herein) to beads to generate free oligonucleotides. These methods can be performed in bulk or partitions or in a 2D array, but as noted herein a benefit is performing these steps in partitions or in a 2D array. In further embodiments described herein, the methods involve starting with oligonucleotides covalently-linked to beads and generating in bulk a population of oligonucleotides that are linked to the beads non-covalently.

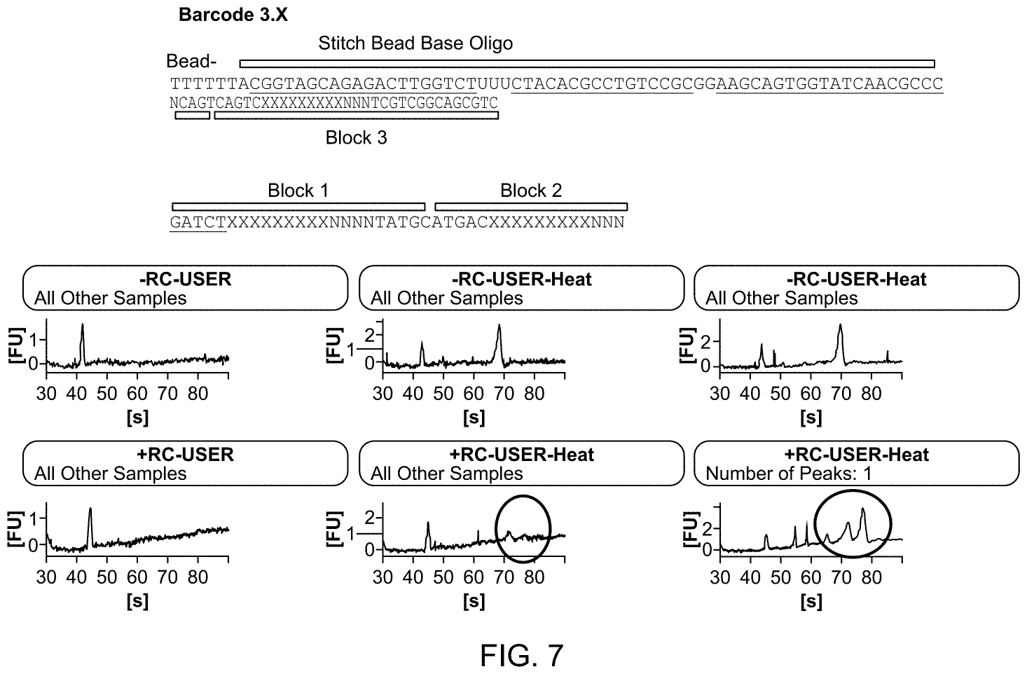

In some embodiments, the nucleic acid strand that will act as a linking oligonucleotide that links the stub sequence (i.e., the oligonucleotide portion remaining covalently linked to the bead following cleavage, see, e.g., FIG. 7, “stitch bead base oligo”) on the bead to the remaining oligonucleotide sequence is initially a separate independent molecule in the mixture. “Separate independent” in this context means that the nucleic acid strand is not covalently linked to the stub sequence or remaining oligonucleotide sequence.

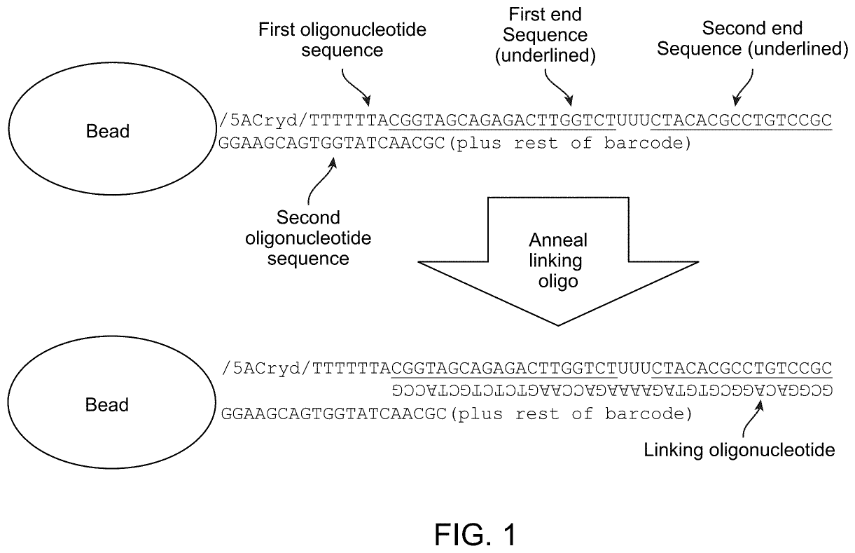

An example of such a configuration is depicted in the upper portion of FIG. 1, in which the top sequence is linked to a bead. Between the underlined sequences are three uracils (U) that represent one possible type of cleavable sequence. The underlined sequences (labelled “first end sequence” and “second end sequence”) are reverse complementary to the separate oligonucleotide directly below, allowing the separate oligonucleotide (also referred to herein as a “linking oligonucleotide”) to hybridize to the depicted first oligonucleotide sequence and the second oligonucleotide sequence, thereby non-covalently linking the two sequences.

While the linking oligonucleotide is hybridized to the first end sequence and the second end sequence of the first and second oligonucleotides, respectively, the intervening cleavable sequence can be cleaved. As discussed in more detail below, the cleavable sequence and ways of cleaving the nucleic a strand containing the cleavable sequence can vary. However, by cleaving the cleavable sequence, the first oligonucleotide sequence and the second oligonucleotide sequence are held together only via non-covalent hybridization of the linking oligonucleotide.

Thus, in some embodiments, one can generate an oligonucleotide in the form of: a first oligonucleotide sequence and the second oligonucleotide sequence held together only via non-covalent hybridization of a linking oligonucleotide as follows. In some embodiments, a mixture of beads is formed, wherein each bead is covalently linked to a “long” oligonucleotide comprising the first oligonucleotide and the second oligonucleotide sequences, wherein the first end sequence of the first oligonucleotide is linked directly, or indirectly via a linker sequence, to the second end sequence of the second oligonucleotide. “Long” in this context simply means that the oligonucleotide comprises both the first and second oligonucleotide sequences as well as an intervening cleavable sequence and thus is longer than either individual sequence alone. The linking oligonucleotide is provided with, or added to the mixture such that the first terminal sequence is hybridized to the first end sequence and the second terminal sequence is hybridized to the second end sequence as they occur in the long oligonucleotide. Hybridization conditions can readily be determined based on the precise sequences hybridization as well as the pH and salt concentration, for example. Once hybridized, the long oligonucleotide strand is cleaved at the cleavable sequence (between the first end sequence and the second end sequence) while the linking oligonucleotide remains intact and links the first oligonucleotide to the second oligonucleotide via hybridization.

The cleavable sequence can be any cleavable sequence that can be targeted enzymatically or otherwise while leaving the rest of the nucleic sequences (e.g., the linking oligonucleotide and the first and second oligonucleotides) in the mixture intact. Cleavage of the cleavable sequence preferably occurs in a bulk solution in which a plurality of beads and their linked oligonucleotides are together in one solution, i.e., prior to partitioning or otherwise spatially distributing them.

In some embodiments, the cleavable sequence comprises one or more uracils. For example, the cleavable sequence can include 1, 2, 3, 4 or more uracils, which can be contiguous. Uracils can be selectively removed and the backbone cleaved (nicked) by contacting with uracil DNA glycosylase and endonuclease VIII, which excises the one or more uracil. Uracil DNA glycosylase and endonuclease VIII is available commercially, for example from New England Biolabs as “USER™” (Uracil-Specific Excision Reagent).

In some embodiments, the cleavable sequence comprises one or more ribonucleotide(s). For example, the cleavable sequence can include 1, 2, 3, 4 or more ribonucleotides, which can be contiguous. This allows one to use an enzyme that selectively cleaves ribonucleotides and does not substantially cleave deoxribonucleotides. For example, in some embodiments, RNAseH is used to specifically cleave at a ribonucleotide in the cleavable sequence.

In some embodiments, the cleavable sequence comprises a restriction enzyme recognition or cleavage site (collectively referred to as a “restriction site”) located between the first oligonucleotide and the second oligonucleotide. In these embodiments, the long oligonucleotide can be cleaved with a restriction enzyme that cleaves the restriction site on the long oligonucleotide without cleaving the linking oligonucleotide. Examples of such enzymes nicking endonuclease. Preferably, the restriction enzyme is selected such that its recognition and/or cleavage site only occurs in the cleavable sequence and does not occur elsewhere in the oligonucleotides in the mixture.

Once the cleavable sequence has been cleaved, the resulting beads and their non-covalently linked oligonucleotides can be distributed to separate partitions or onto a 2D array and then the oligonucleotides can be released from the beads in the partitions. In some embodiments, the non-covalently linked oligonucleotides and beads are generated by the user (e.g., as discussed above), whereas in other embodiments, they are provided to the user (e.g., from a commercial supplier). In either option, the user can distribute the beads and non-covalently linked oligonucleotides into partitions or onto a 2D array (e.g., a surface). Distributing the beads and non-covalently linked oligonucleotides into partitions can be achieved by any methods available. For example, partitions can be pre-formed, optionally with other agents and optionally target nucleic acids from a biological sample and the beads and non-covalently linked oligonucleotides can be injected or otherwise introduced into the partitions. Methods and compositions for delivering reagents to one or more partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

In other embodiments, for example in which the partitions are droplets, one can form droplets as an emulsion with an immiscible fluid such as oil such that the bulk solution forms droplets that contain the beads and non-covalently linked oligonucleotides, optionally with other reagents and/or a sample nucleic acid. Methods of emulsion formation are described, for example, in published patent applications WO 2011/109546 and WO 2012/061444,

Distribution of beads into partitions (e.g., such as droplets) can be dictated by a Poisson distribution, in some embodiments. Depending on the end use, the average number of beads per partition can be less than 1 (e.g., 0.2-0.9), 1, or more than 1 (e.g., 1-3 or more). In some embodiments, it is desirable to avoid multiple beads in a partition and in these cases many partitions may be left empty such that a majority of partitions that contain a bead only contain one bead. In other embodiments, e.g., in which deconvolution methods can be used to decipher sequencing results where multiple beads occur in a single partition, more beads can be loaded on average per partition, with deconvolution being used after to resolve sequencing results. See, e.g., PCT/US2017/012618; PCT/US2019/015638; PCT/US2020/36699. In some embodiments, the beads and associated oligonucleotides are partitioned into at least 500 partitions, at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions.

In some embodiments, after distribution of the beads and non-covalently linked oligonucleotides into partitions, the non-covalently linked oligonucleotides can be released from the beads, e.g., prior to use of the oligonucleotides to attach barcodes, perform primer extension to perform other uses of the oligonucleotides. Release of the non-covalently linked oligonucleotides from the beads can involve exposing the non-covalently linked oligonucleotides to conditions such that the hybridized portions disassociate. This can be achieved, for example by raising the temperature above the melting temperature Tm of the hybridizing sequences. In embodiments in which there is a linker oligonucleotide, the first terminal sequence of the linker sequence and the first end sequence of the first oligonucleotide are hybridized and have a first melting temperature (Tm) and (ii) the second terminal sequence of the linker oligonucleotide and second end sequence are hybridized and have a second Tm such that the linking oligonucleotide links the first oligonucleotide to the second oligonucleotide. In these embodiments, the reaction mixture temperature can be raised above the melting temperature of at least one of the first or second Tm. In some embodiments, the Tms are within 10 or 5 degrees such that raising the mixture temperatures above one of the Tms also raise the temperature above the other Tm such that both sets of hybridizing sequences are disassociated.

Prior to lowering the temperature below the Tm (or Tms), a blocking oligonucleotide can be introduced into the mixture. The blocking oligonucleotide can be introduced into the mixture much earlier in the process, e.g., any time in the above sequence of events, including upon first forming the mixture, but should be present before the temperature is lowered. As discussed in more detail below, in embodiments in which a hairpin oligonucleotide is employed and in embodiments in which the blocking competition function is provided by an intramolecular sequence, a blocking oligonucleotide need not be provided at all. However, when present, the blocking oligonucleotide will compete for binding to one of the oligonucleotide to be released or the remaining stub sequence present on the bead. In either case, by annealing to one of these sequences, the released oligonucleotide will remain free of the bead and will not re-anneal. In some embodiments, the blocking oligonucleotide will be provided at a higher concentration than the concentration of the released oligonucleotides, thereby allowing the blocking oligonucleotide to better compete for binding. In some embodiments, the blocking oligonucleotide can have one or more non-natural nucleotide such that the affinity for the sequence to which it anneals is strong (lower Kd) than the released oligonucleotide.

The blocking oligonucleotide can be configured in four alternatives to compete for and prevent re-annealing of the released oligonucleotide back to the stub sequence on the bead:
(a) In some embodiments, the blocking oligonucleotide comprises a sequence that is reverse complementary to the first end sequence but does not comprise a sequence of more than 2, 3, 4, or 5 contiguous nucleotides reverse complementary to the second end sequence (i.e., so that it does not “bridge” the first and second oligonucleotides), such that the blocking oligonucleotide competes with the linking oligonucleotide for hybridization to the first oligonucleotide, allowing the second oligonucleotide to remain released from the bead.
(b) In some embodiments, the blocking oligonucleotide comprises a sequence that is reverse complementary to the second end sequence but does not comprise a sequence of more than 2, 3, 4, or 5 contiguous nucleotides reverse complementary to the first end sequence, such that the blocking oligonucleotide competes with the linking oligonucleotide for hybridization to the second oligonucleotide, allowing the second oligonucleotide to remain released from the bead.
(c) In some embodiments, the blocking oligonucleotide comprises a sequence that is the first end sequence but does not comprise a sequence of more than 2, 3, 4, or 5 contiguous nucleotides in the second end sequence, such that the blocking oligonucleotide competes with the linking oligonucleotide for hybridization to the first oligonucleotide, allowing the second oligonucleotide to remain released from the bead.
(d) In some embodiments, the blocking oligonucleotide comprises a sequence that is the second end sequence but does not comprise a sequence of more than 2, 3, 4, or 5 contiguous nucleotides that is the first end sequence, such that the blocking oligonucleotide competes with the linking oligonucleotide for hybridization to the second oligonucleotide, allowing the second oligonucleotide to remain released from the bead.

### Hairpin

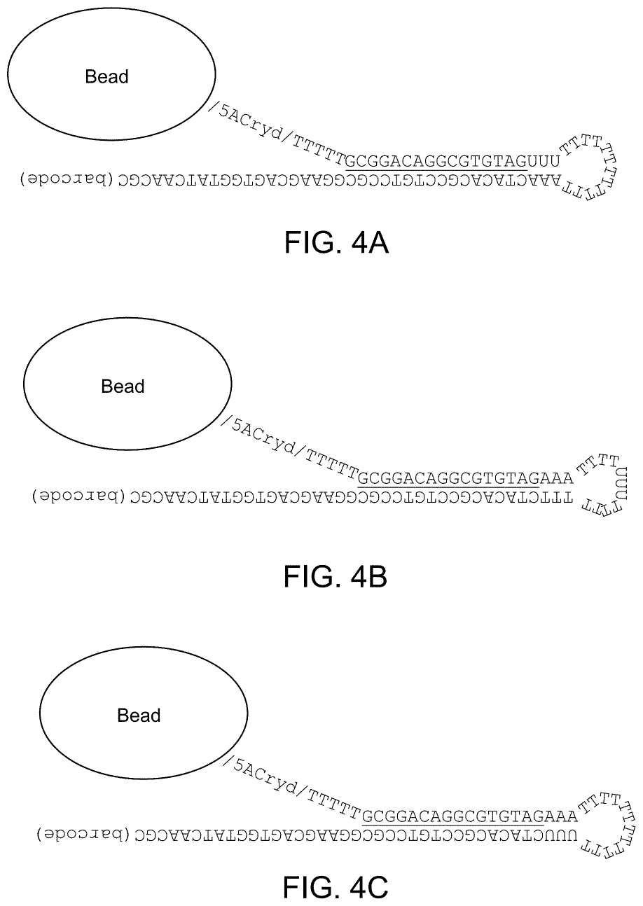

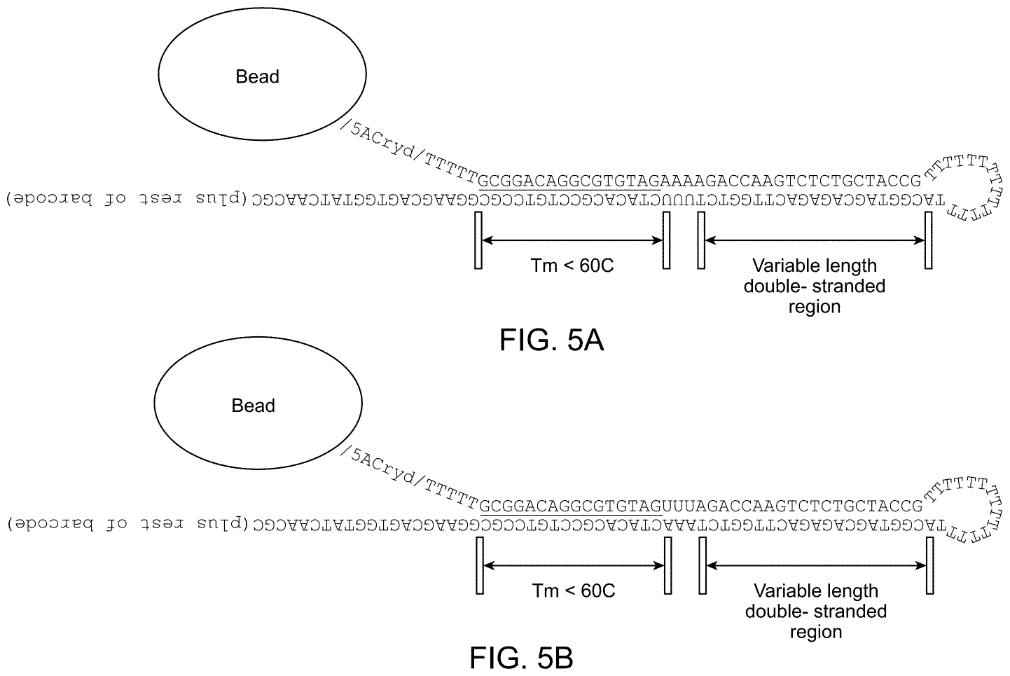

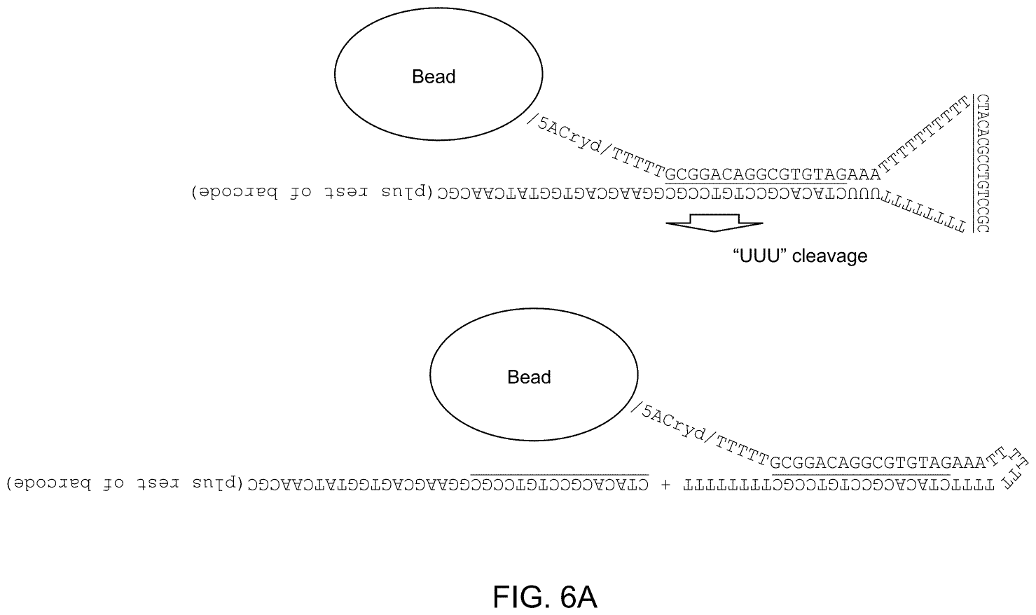

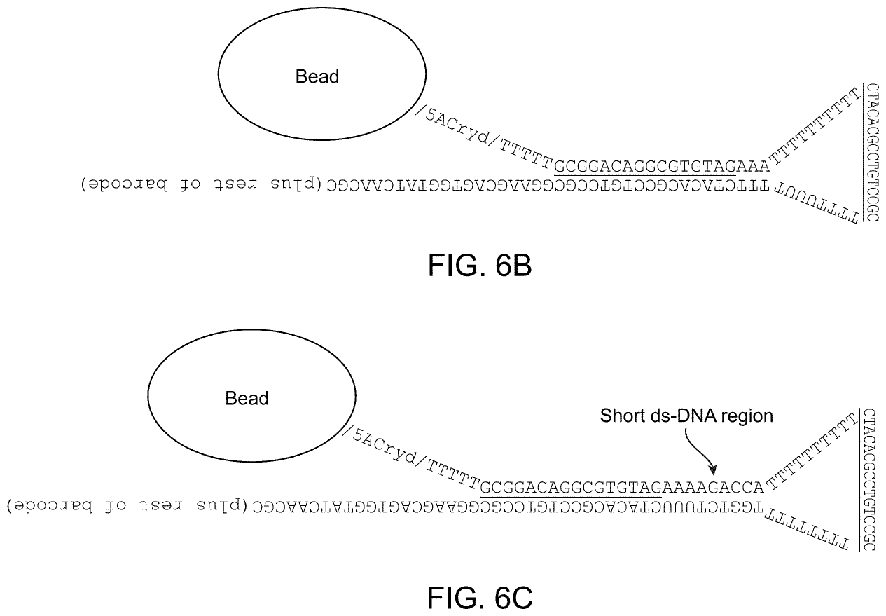

In some embodiments, a separate linking oligonucleotide is not employed or required because the initial oligonucleotide linked to the bead is a hairpin with a self-complementary sequence and thus is linked to itself. In some embodiments, the bead is covalently linked to a hairpin oligonucleotide comprising 5′ to 3′ a reverse complement of a first sequence, a loop sequence comprising a cleavable sequence, a first copy of the first sequence, and a second sequence, wherein the reverse complement of the first sequence is hybridized to the first copy of the first sequence, and the second sequence is at the 3′ end of the hairpin oligonucleotide. An example of this embodiments can be found in for example, FIG. 4B. In some embodiments, the loop sequence further comprises a second copy of the first sequence (see, e.g., FIG. 6) and in some embodiments, the loop sequence does not comprise a second copy of the first sequence (e.g., FIG. 4A-C and FIG. 5A-B). In some embodiments the cleavable sequence is located within the reverse complement of the first sequence (e.g., FIG. 5B), 5′ of the loop sequence (e.g., FIG. 4A), within the loop sequence (FIG. 4B), 3′ of the loop sequence (FIG. 4C), and/or within the first sequence (FIG. 5A). When the loop sequence comprises a second copy of the first sequence, the cleavable sequence is located 3′ of the second copy of the first sequence (FIG. 6A-C).

In either embodiment, a plurality of beads linked to hairpin oligonucleotides can be provided in bulk and the cleavable sequence can be cleaved to generate a portion of the cleaved hairpin oligonucleotides comprising the reverse complement of a first sequence still covalently linked to the bead and a second portion of the cleaved hairpin oligonucleotides comprising the first copy of the first sequence, and a second sequence. The second portion in these embodiments is no longer covalently linked to the bead but is non-covalently linked via hybridization of the first copy of the first sequence to the reverse complement of a first sequence. See, e.g., FIG. 4A-C. The cleavable sequence and its cleavage can be achieved as discussed above.

Once cleaved, e.g., in a bulk mixture, the beads and their non-covalently-attached portions can be distributed to partitions or onto a 2D array. One distributed, the non-covalently-attached oligonucleotide portions can be released (e.g., in the partitions or on the 2D array), for example by raising the temperature above the Tm of the first copy of the first sequence to the reverse complement of a first sequence. As discussed above for a different configuration, prior to lowering the temperature below the Tm, a blocking oligonucleotide can be introduced into the mixture. The blocking oligonucleotide can be introduced into the mixture much earlier in the process, e.g., any time in the above sequence of events, including upon first forming the mixture, but should be present before the temperature is lowered. The blocking oligonucleotide will compete for binding to one of: (1) the first copy of the first sequence or (2) the reverse complement of a first sequence. In either case, by annealing to one of these sequences, the released oligonucleotide will remain free of the bead and will not re-anneal. In some embodiments, the blocking oligonucleotide will be provided at a higher concentration than the concentration of the released oligonucleotides, thereby allowing the blocking oligonucleotide to better compete for binding. In some embodiments, the blocking oligonucleotide can have one or more non-natural nucleotide such that the affinity for the sequence to which it anneals is strong (lower Kd) than the released oligonucleotide.

In yet another embodiment, the hairpin oligonucleotide comprises a loop sequence that further comprises a second copy of the first sequence (see, e.g., FIG. 6). This configuration can be employed as described above for other hairpin oligonucleotide configurations, but need not utilize the blocking oligonucleotide because the second copy of the first sequence acts to compete with re-hybridization (re-annealing) of the first copy of the first sequence to the reverse complement of a first sequence. Moreover, because this reaction is intramolecular, the second copy of the first sequence being covalently linked to the reverse complement of a first sequence, the second copy of the first sequence should out-compete the released portion of the hairpin oligonucleotide comprising the first copy of the first sequence, allowing the released portion to remain free in solution. See, e.g., FIG. 6A

In any of the embodiments described herein, the oligonucleotides linked to the beads can comprise one or more barcode nucleotide sequences. In some embodiments, the oligonucleotides include a barcode sequence that is unique to the bead to which it is attached and thus can be used to distinguish oligonucleotides from different beads, e.g., after the oligonucleotides are released and used to generate sequencing reads. Additional barcodes, such as but not limited to, unique molecule identifiers (UMIs) or sample-specific barcodes can also be included in the oligonucleotide sequence, i.e., in the portion of the oligonucleotide that is ultimately released in the partitions or onto the 2D array.

Any bead of useful size and composition for delivery to partitions or 2D array can be used The particle or bead can be any particle or bead having a solid support surface. Solid supports suitable for particles include controlled pore glass (CPG)(available from Glen Research, Sterling, Va.), oxalyl-controlled pore glass (See, e.g., Alul, et al., Nucleic Acids Research 1991, 19, 1527), TentaGel Support—an aminopolyethyleneglycol derivatized support (See, e.g., Wright, et al., Tetrahedron Letters 1993, 34, 3373), polystyrene, Poros (a copolymer of polystyrene/divinylbenzene), or reversibly cross-linked acrylamide. Many other solid supports are commercially available and amenable to the present invention. In some embodiments, the bead material is a polystyrene resin or poly(methyl methacrylate) (PMMA). The bead material can be metal.

In some embodiments, the particle or bead comprises hydrogel or another similar composition. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 20020009591; 20130022569; 20130034592; and International Patent Publication Nos. WO1997030092; and WO2001049240. Additional compositions and methods for making and using hydrogels, such as barcoded hydrogels, include those described in, e.g., Klein et al., Cell, 2015 May 21; 161(5):1187-201.

The solid support surface of the bead can be modified to include a linker for attaching barcode oligonucleotides. The linkers may comprise a cleavable moiety. Non-limiting examples of cleavable moieties include a disulfide bond, a dioxyuridine moiety, and a restriction enzyme recognition site.

Oligonucleotides can be linked to beads as desired. Methods of linking oligonucleotides to beads are described in, e.g., WO 2015/200541. In some embodiments, the oligonucleotide configured to link a hydrogel bead to the barcode is covalently linked to the hydrogel. Numerous methods for covalently linking an oligonucleotide to one or more hydrogel matrices are known in the art. As but one example, aldehyde derivatized agarose can be covalently linked to a 5′-amine group of a synthetic oligonucleotide.

One delivered to the partitions or 2D arrays and in the presence of a target (e.g., sample) nucleic acid, the oligonucleotides released from the beads can be used to perform primer extension or other hybridization-based reactions. In some embodiments, the 3′ end sequences of the oligonucleotides anneal directly to the target nucleic acids. For example, if the target nucleic acids are mRNA, the 3′ end sequence can be a poly dT sequence (e.g., 6-20 contiguous dT nucleotides), or the 3′ end can include randomer (e.g., random sequences of 6- or more nucleotides) to randomly prime targets, or the 3′ end sequences can be gene-specific to specifically amplify one or more target nucleic acids. In some embodiments, the 3′ sequence of the oligonucleotides anneals to a universal sequence on the target nucleic acids. For example, tagamentation can result in insertion of an adaptor sequence to the end of fragmented nucleic acids and the 3′ end can be reverse complementary to the adaptor sequence.

In some embodiments, once the oligonucleotides are released into the partitions or onto a 2D array, they can be used for example to amplify sample nucleic acids in the partitions or on the 2D array by extension of the 3′ ends of the oligonucleotides, e.g., by a polymerase to copy portions of the sample nucleic acids, thereby tagging the sample nucleic acids with the oligonucleotide sequence, which can preferably include one or more barcode sequence (e.g., a bead-specific barcode sequence) as described herein. The amplified sequences can then be used as desired. In some embodiments, the amplified cDNAs can be cloned into a vector or otherwise be formulated into a cDNA library, which can optionally be stored and replicated as desired.

In some embodiments, the amplified nucleic acids can be nucleotide sequenced. Once the nucleic acids have been tagged with the oligonucleotides, the tagged nucleic acids can be prepared for nucleotide sequencing as desired. For example, universal priming sequences can be added on both ends of the tagged sequences (one universal priming sequence Any method of nucleotide sequencing can be used as desired so long as at least some of the DNA segments sequence and the barcode sequence is determined. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/084132, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

The practice of the present invention can employ conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999-2010) Current Protocols in Molecular Biology, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag; Sambrook et al., Molecular Cloning: A Laboratory Manual (2nd ed.) (1989).

Any type of partitions can be used with the methods and compositions described herein. In some embodiments, the beads can be inserted into partitions (e.g., droplets or wells). In some embodiments, the beads are encapsulated into aqueous droplets in a water-in-oil emulsion. Methods and compositions for partitioning a sample are described, for example, in published patent applications WO 2010/036,352, US 2010/0173,394, US 2011/0092,373, and US 2011/0092,376, the contents of each of which are incorporated herein by reference in the entirety. The plurality of mixture partitions can be in a plurality of emulsion droplets, or a plurality of wells, etc. The partitions can be picowells, nanowells, or microwells. In some embodiments, there are at least e.g., 100,000 wells, or 200,000 wells e.g., 100,000-500,000 wells. Exemplary wells can have a volume capacity of e.g., 10-50 picoliters. Exemplary wells include those as described in U.S. Patent Publication No. US2021/0283608. The mixture partitions can be pico-, nano-, or micro-reaction chambers, such as pico, nano, or microcapsules. The mixture partitions can be pico-, nano-, or micro-channels. The mixture partitions can be droplets, e.g., emulsion droplets.

As described herein, in some embodiments, the beads and non-covalently linked oligonucleotides are distributed on a 2D array. Methods of generating arrays of oligonucleotides with known sequences are known and can be used to generate the arrays described herein. See, for example, US2021/0332351 and US2020/0299322 or as otherwise described by for example DNA Script or Twist Biosciences. These methods, can for example, provide for spatially addressable oligonucleotides on a planar array, meaning that the oligonucleotide sequences at each spot on the array are known. In some embodiments, such planar supports have a plurality of sites comprising at least 256 sites, at least 512 sites, at least 1024 sites, at least 5000 sites, at least 10,000 sites, at least 25,000 sites, or at least 100,000 sites and as many as 10,000,000 sites. In some embodiments, the discrete site at which synthesis of spots take place each has an area in the range of from 0.25 μm² to 1000 μm², or from 1 μm² to 1000 μm², or from 10 μm² to 1000 μm², or from 100 μm² to 1000 μm². In some embodiments, the amount of polynucleotides synthesized at each spot is at least 10⁻⁶ fmol, or at least 10⁻³ fmol, or at least 1 fmol, or at least 1 μmol, or the amount of polynucleotide synthesized at each spot is in the range of from 10⁻⁶ fmol to 1 fmol, or from 10⁻³ fmol to 1 fmol, or from 1 fmol to 1 μmol, or from 10⁻⁶ pmol to 10 pmol, or from 10⁻⁶ pmol to 1 pmol. In some embodiments, the number of polynucleotides synthesized at each spot is in the range of from 1000 molecules to 106 molecules, or from 1000 molecules to 109 molecules, or from 1000 molecules to 1012 molecules. In some embodiments, the array is on a flow cell.

In some embodiments, the methods described herein can be used for spatial profiling. Spatial profiling is a method for highly multiplex spatial profiling of proteins or RNAs suitable for use on formalin-fixed, paraffin-embedded (FFPE) samples. See, e.g., Beecham, Methods Mol Biol. 2055:563-583 (2020). In some embodiments, the methods described herein allow to improved spatial profiling methods by using in situ tagmentation in a fixed (e.g., FFPE) tissue sample. Following tagmentation of nucleic acids in situ in a tissue sample, the tissue can be contacted with beads linked to clonal barcoding oligonucleotides. Alternatively, the tissue can be contacted with released barcoding oligonucleotides from beads in near proximity to the tissue.

### EXAMPLES

### Example 1 (Prophetic)

Twenty-three micron diameter clonal barcode gel beads with hairpin oligos terminated with an oligo dT sequence and comprising 3 uracil nucleotides in the loop sequence are incubated with USER enzyme comprising uracil DNA glycosylase and endonuclease VIII at 37° C. for 30 min in bulk. The uracil nucleotides are cleaved from the clonal oligonucleotides. Hybridization of the sequence 5′ upstream of the loop sequence and 3′ downstream of the loop sequence, whereby the loop sequence contains the cleaved uracil nucleotides, is maintained due to a Tm of their double-stranded hybridized sequence of approximately 60° C. In consequence, even though the loop sequence are cleaved, the 3′ cleaved oligo containing the barcode and the oligo dT priming sequence remain noncovalently attached to the bead through hybridization with the remaining oligo attaching to the bead. A blocker oligonucleotide complementary to the hybridized portion of the remaining oligonucleotide attached to the bead is added to a concentration of 5 μM in bulk. The beads and blocker oligonucleotides are loaded together with cells into microwells 25 micron in diameter such that only one bead can fit into the well. Cells are loaded at lambdas of <0.06 such that the majority of wells have either one or none cells. Warm start reverse transcriptase, and a buffer containing 0.5% NP-40 to lyse cells is flowed across the space above the microwells. After allowing for diffusion into the microwells, the wells are sealed with oil by flowing oil above the microwells. The temperature of the chip is then raised to 70° C., which is not only sufficient to warmstart the reverse transcriptase, but also to denature the cleaved 3′ oligonucleotide from the remaining oligo attached to the bead. The temperature is then decreased to 42° C. allowing for the hybridization of the blocker to the remaining oligo on the gel bead. Due to the hybridization of the blocker oligo, the released 3′ barcode oligo remain in solution. The reaction is held at 42° C. for 60 min. As the cells have been lysed with lysis reagent, reverse transcription through primed 3′ cleaved barcode dT oligonucleotides occurs. After the reverse transcription step, the oil is removed over the microwells and the cDNA retrieved from the chip. Sample prep and sequencing ensues followed by single cell analysis grouping the barcode sequences and attributing them to individual partitions and single cells.

### Example 2

The base bead oligonucleotide for examples 2-4 was as follows:

TTTTTTACGGTAGCAGAGACTTGGTCTUUUCTACAC
GCCTGTCCGCGGAAGCAGTGGTATCAACGC

Following creation of gel beads with the base bead oligonucleotide integrated into the matrix, barcode sequences together with template annealing sequence were added resulting in a full length bead oligonucleotide sequence.

The following splint oligonucleotide, splint oligonucleotide 1, was used in this experiment:

GCGGACAGGCGTGTAGAAAAGACCAAGTCTCTGCTACCG

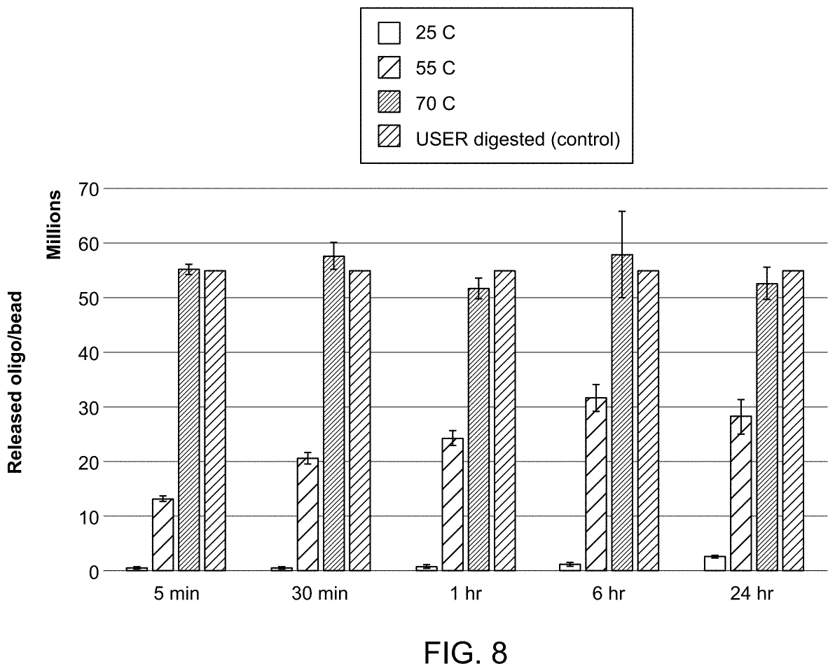

An excess, i.e. 2 nmole, of splint oligonucleotide 1 having a Tm of 62° C., was annealed to the base oligonucleotide by mixing the splint oligonucleotide with 500, 000 base beads followed by an incubation at 95 C for 5 min and then a slow cooling to RT by removing the incubation tube from the heat source. Post oligonucleotide annealing, USER from NEB was added to the bead mix followed by incubation at 37° C. to digest the U's in the base oligonucleotide. The beads were then washed to remove the USER enzyme. The bead slurry was subsequently distributed into different tubes and the tubes were incubated at the followed temperatures: 25° C., 55° C., and 70° C. After mixing the bead slurry to assure homogenization, a small aliquot of the supernatant was removed at the following time points and subject to ddPCR quantification: 5 min, 30 min, 60 min, 6 hr, and 24 hr. As a positive control, a splint minus condition was performed in parallel to determine how much oligonucleotide could be maximally released. Since the Tm's of the sequence flanking the Us was higher than 25° C., the complex remained stable with minimal release of the oligonucleotide detected for the 25° C. across all time points, reaching approximately 5% of control after 24 hr. At the 55 C incubation temperature the release grew from approximately 20% of control to 50% at the maximal time. At 70° C., the amount of bead oligonucleotide released was approximately equal to the positive control at all time points measured, as expected since at this temperature the Tm of the splint oligonucleotide is greatly exceeded. See FIG. 8. These data support the following conclusions: The splint oligonucleotide annealed completely to the base oligo. USER cleavage was productive in removing the U's in the base oligonucleotide splint complex. At low temperature incubations below the Tm, the cleaved base oligonucleotide splint complex was stable. At high temperature incubations, maximal bead oligonucleotide was released from the cleaved base-splint oligonucleotide complex.

### Example 3

The following splint oligonucleotide, splint oligonucleotide 2, was used in this experiment:

GCAGGCGTGTAGAAAAGACCAAGTCTCTG

Splint oligonucleotide #2, with a Tm of 50° C., was annealed to the base oligonucleotide attached to a mixture of 55 million beads. The annealing step was achieved by the same method as stated in Example 2. USER was then added and the mixture was incubated at 37° C. to digest the U's in the base oligonucleotide. Following splint—base oligonucleotide complex formation and USER cleavage, the beads were washed and approximately 10,000 beads were removed from the mixture and aliquoted. Approximately 10,000 cells were added to the aliquoted beads, followed by reverse transcription master mix containing reverse transcriptase and lysis reagents. The contents were mixed well. The reaction mixture was incubated at 50° C. to enable some oligonucleotide release (with a splint Tm of 50° C., only approximately 50% of oligonucleotides are expected to be released provided that USER cleavage was efficient) and reverse transcription. An aliquot of the mixture was removed and GAPDH bead oligonucleotide templated cDNA molecules were quantified by ddPCR. This was accomplished by using ddPCR primers targeting the bead oligonucleotide common 5′ sequence on one side of the amplicon and GAPDH sequences on the other side. In parallel, the same procedure was performed on beads lacking the splint oligonucleotide. This constituted a positive control as all bead barcode oligonucleotides are expected to be maximally released. ddPCR measurements showed that the splint oligonucleotide condition achieved approximately 40% of GAPDH bead oligonucleotide cDNA conversion compared to the control, as expected due to a similar Tm of the splint oligonucleotide bead complex as that of the reverse transcription incubation temperature. See,

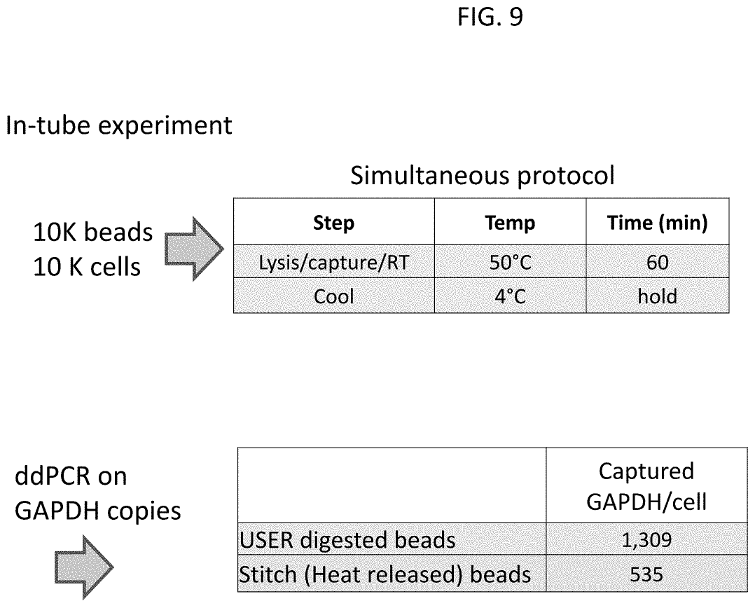

FIG. 9. These data indicates that USER digestion of the splint base bead oligonucleotide complex was efficient and that approximately half of oligonucleotides were released to perform reverse transcription in the mixture. Moreover, the data indicates that the reaction mixture is a feasible solution to simultaneously release the oligonucleotide, lyse the cell, and perform reverse transcription in solution.

### Example 4

The following splint oligonucleotide, splint oligonucleotide 3, with a Tm of 68° C., was used in this experiment:

CGCGGACAGGCGTGTAGAAAAGACCAAGTCTCTGCTACCGTAAA

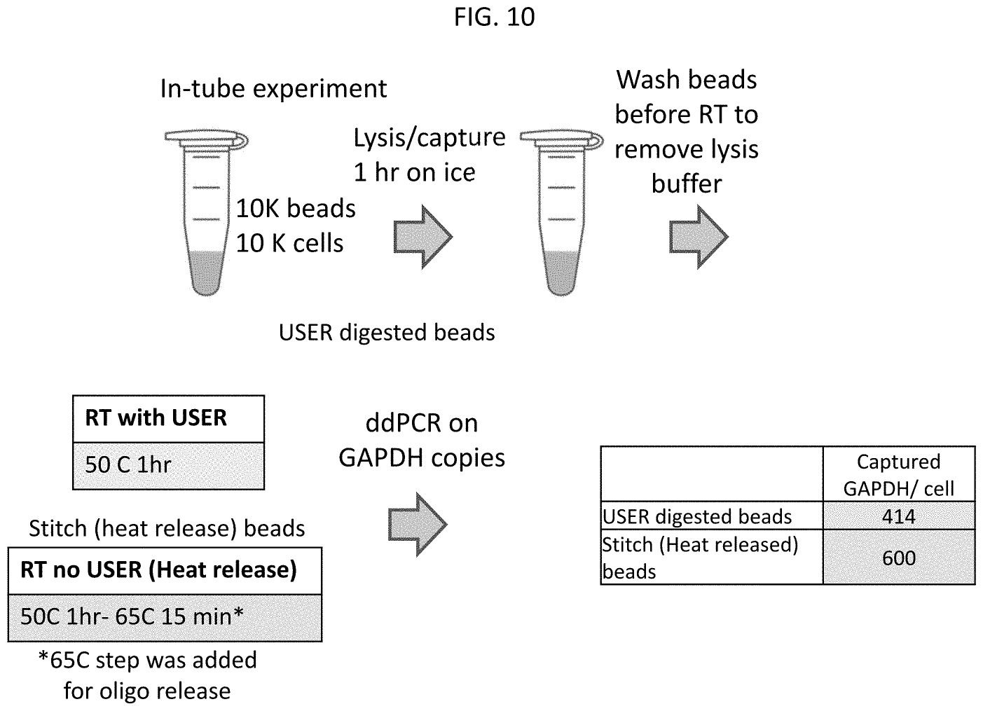

Using splint oligonucleotide 3, a similar procedure to that described in Example 3 was followed in Example 4 up to the step of aliquoting 10 000 beads and cells. Rather than performing oligonucleotide release, lysis and reverse transcription together, these operations were performed in series. USER was first added to cleave the U's in the bead oligonucleotide. Cells were then lysed by added lysis reagent and incubating on ice for 1 hr, simultaneously allowing for RNA hybridization to bead oligonucleotides. Beads were then washed to remove the USER and lysis reagents followed by the addition of reverse transcription master mix and reverse transcriptase. The mix was incubated at 50° C. for 1 hr to activate reverse transcription followed by a 15 min step at 65° C. to release oligonucleotides. GAPDH was measured by ddPCR as described in Example 3. As a positive control, splint oligos were omitted from the procedure and USER was added during the reverse transcription step. ddPCR of bead oligonucleotide tagged cDNA showed that the in solution reverse transcription driven by released bead oligonucleotide was as efficient as the positive control. See, FIG. 10. This demonstrates that oligonucleotides were stable post USER cleavage and that subsequent oligonucleotide release with heat was effective. Together these data indicate the functionality of stitch bead oligonucleotides and their release by heat in a sequential cell lysis—reverse transcription methodology.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of releasing an oligonucleotide from a bead, the method comprising,
(i) providing a reaction mixture comprising:
a plurality of beads, each bead covalently linked to a first oligonucleotide comprising a first end sequence,
a second oligonucleotide comprising a second end sequence; and
a linking oligonucleotide comprising (i) a first terminal sequence that is reverse complementary to the first end sequence and (ii) a second terminal sequence that is reverse complementary to the second end sequence,
wherein the first terminal sequence and first end sequence are hybridized and have a first melting temperature (Tm) and (ii) the second terminal sequence and second end sequence are hybridized and have a second Tm such that the linking oligonucleotide links the first oligonucleotide to the second oligonucleotide;
(ii) raising the temperature of the reaction mixture higher than at least one of the first and second Tm such that the first oligonucleotide and the second oligonucleotide are disassociated from at least one end of the linking oligonucleotide;
(iii) lowering the temperature of the reaction mixture below the first and second Tm,
wherein after the raising and before the lowering the reaction mixture further comprises a blocking oligonucleotide comprising either:
(a) a sequence that is reverse complementary to the first end sequence but does not comprise a sequence of more than 3 contiguous nucleotides reverse complementary to the second end sequence, such that the blocking oligonucleotide competes with the linking oligonucleotide for hybridization to the first oligonucleotide, allowing the second oligonucleotide to remain released from the bead, or
(b) a sequence that is reverse complementary to the second end sequence but does not comprise a sequence of more than 3 contiguous nucleotides reverse complementary to the first end sequence, such that the blocking oligonucleotide competes with the linking oligonucleotide for hybridization to the second oligonucleotide, allowing the second oligonucleotide to remain released from the bead or
(c) a sequence that is the first end sequence but does not comprise a sequence of more than 3 contiguous nucleotides in the second end sequence, such that the blocking oligonucleotide competes with the linking oligonucleotide for hybridization to the first oligonucleotide, allowing the second oligonucleotide to remain released from the bead, or
(d) a sequence that is the second end sequence but does not comprise a sequence of more than 3 contiguous nucleotides that is the first end sequence, such that the blocking oligonucleotide competes with the linking oligonucleotide for hybridization to the second oligonucleotide, allowing the second oligonucleotide to remain released from the bead.

2. The method of claim 1, wherein the first end sequence is a 3′ end sequence.

3. The method of claim 1, wherein the first end sequence is a 5′ end sequence.

4. The method of claim 1, wherein the second oligonucleotide has a barcode sequence, wherein individual beads comprise clonal copies of the second oligonucleotide and wherein the barcode sequence for individual beads are unique such that the barcode distinguishes the bead from other beads in the plurality.

5. The method of claim 4, wherein the 3′ end of the second oligonucleotide comprises a target-specific sequence.

6. The method of claim 4, wherein the 3′ end of the second oligonucleotide comprises a universal tag sequence.

7. The method of claim 4, wherein the 3′ end of the second oligonucleotide comprises at least 4 contiguous thymines.

8. The method of claim 1, wherein the providing (i) comprises forming a mixture of beads, wherein each bead is covalently linked to a long oligonucleotide comprising the first oligonucleotide and the second oligonucleotide, wherein the first end sequence of the first oligonucleotide is linked directly, or indirectly via a linker sequence, to the second end sequence of the second oligonucleotide, and wherein long oligonucleotides on different beads are distinguishable by a different barcode sequence in the long oligonucleotide; and
hybridizing the linking oligonucleotide to the long oligonucleotide such that the first terminal sequence is hybridized to the first end sequence and the second terminal sequence is hybridized to the second end sequence; and
cleaving the long oligonucleotide between the first end sequence and the second end sequence while the linking oligonucleotide remains intact and links the first oligonucleotide to the second oligonucleotide.

9. The method of claim 8, wherein the linker sequence comprises one or more uracil nucleotide and the cleaving comprises contacting the long oligonucleotide with uracil DNA glycosylase and endonuclease VIII, thereby excising the one or more uracil.

10. The method of claim 8, wherein the linker sequence comprises one or more ribonucleotide and the cleaving comprises cleaving the linker sequence in a ribonucleotide-specific manner using RNAseH.

11. The method of claim 8, wherein a restriction site is located between the first oligonucleotide and the second oligonucleotide and the cleaving comprises contacting the long oligonucleotide with a restriction enzyme that cleaves the restriction site on the long oligonucleotide without cleaving the linking oligonucleotide using a nicking endonuclease.

12. The method of any one of claims 9-11, wherein the blocking oligonucleotide is added to the reaction mixture following the cleaving of the long oligonucleotide between the first end sequence and the second end sequence.

13. The method of any one of claims 1-11, wherein the concentration of the blocking oligonucleotide in the reaction mixture is higher than the concentration of the linker oligonucleotide in the reaction mixture.

14. The method of any one of claims 1-13, wherein the affinity (Kd) of the blocking oligonucleotide for the first sequence is lower than the affinity of the linker oligonucleotide for the first sequence.

15. The method of any one of claims 1-14, further comprising distributing the reaction mixture into a plurality of partitions after the providing (i) and before the raising (ii), wherein different beads of the plurality are delivered into different partitions.

16. The method of claim 15, wherein the partitions are microwells, nanowells or droplets.

17. The method of any one of claims 1-14, further comprising distributing the reaction mixture onto a 2D array after the providing (i) and before the raising (ii), wherein different beads of the plurality are delivered onto different locations on the 2D array.

18. A method of forming a cleaved oligonucleotide linked to a bead, the method comprising
forming a mixture of beads, wherein each bead is covalently linked to a long oligonucleotide comprising a first oligonucleotide and a second oligonucleotide, wherein a first end sequence of the first oligonucleotide is linked directly, or indirectly via a linker sequence, to a second end sequence of the second oligonucleotide, and wherein long oligonucleotides on different beads are distinguishable by a different barcode sequence in the long oligonucleotide; and
hybridizing a linking oligonucleotide to the long oligonucleotide, wherein the linking oligonucleotide comprises (i) a first terminal sequence that is reverse complementary to the first end sequence and (ii) a second terminal sequence that is reverse complementary to the second end sequence, wherein the hybridizing results in the first terminal sequence hybridized to the first end sequence and the second terminal sequence hybridized to the second end sequence; and
cleaving the long oligonucleotide between the first end sequence and the second end sequence while the linking oligonucleotide remains intact and links the first oligonucleotide to the second oligonucleotide.

19. The method of claim 18, wherein the linker sequence comprises one or more uracil nucleotide and the cleaving comprises contacting the long oligonucleotide with uracil DNA glycosylase and endonuclease VIII, thereby excising the one or more uracil.

20. A mixture comprising a plurality of beads, wherein each bead is covalently linked to a hairpin oligonucleotide comprising 5′ to 3′ a reverse complement of a first sequence, a loop sequence a first copy of the first sequence, and a second sequence, wherein the reverse complement of the first sequence is hybridized to the first copy of the first sequence, and the second sequence is at the 3′ end of the hairpin oligonucleotide,
wherein a cleavable sequence is located in the reverse complement of the first sequence, in the loop sequence, or in the first copy of the first sequence,
wherein the first sequence has a barcode sequence, wherein individual beads comprise clonal copies of the first sequence and wherein the barcode sequence for individual beads are unique such that the barcode distinguishes the bead from other beads in the plurality.
